(19) United States
(12) Patent Application Publication
(10) Pub. No.: US 2007/0060795 A1
(43) Pub. Date: Mar. 15, 2007

(54) Lighted surgical retractors with LED illumination light engines

(75) Inventors: Alex Vayser, Mission Viejo, CA (US); Kenneth Trauner, San Francisco, CA (US)

Correspondence Address: Crockett & Crockett, Suite 400, 24012 Calle De La Plata, Laguna Hills, CA, 92653, US

(73) Assignee: Spotlight Surgical, Inc.

(21) Appl. No.: 11/227,500

(22) Filed: Sep. 14, 2005

(51) Int. Cl. A61B 1/32

(52) U.S. Cl. 600/245

(57) ABSTRACT

Lighting systems for surgical retractors.

### FIELD OF THE INVENTIONS

The inventions described below relate the field of illuminated surgical retractors.

### BACKGROUND OF THE INVENTIONS

Existing technology for illumination during surgical/medical procedures is currently limited to overhead illumination. This illumination comes from either overhead lighting or head mounted fiber optic systems. Traditional overhead lighting systems face numerous limitations. Direct exposure of the field from the overhead source is required. Changes in patient or surgeon positioning may interfere with the light source. Frequent adjustments provide an inconvenience for the surgeon and disrupt the surgical flow. Overhead lighting is frequently inadequate for surgery in deeper cavities where more intense focused illumination may be required. In addition, the alignment of the surgeon's head frequently interferes with the remote illumination and prevents light from reaching the field. Head mounted fiber optic systems are used frequently for more limited surgical exposures however, these devices have numerous limitations. First, the surgeon is tethered by the light cord attached to the headset, limiting mobility in the operating room. Second, the devices are associated with head and neck fatigue with frequent or more prolonged use. Third, the devices require the surgeon to maintain a steady head and neck position to provide a constant and steady illumination of the field. Fourth, the use of remote light sources and fiber bundles introduces tremendous inefficiencies into the system. A typical ten foot long cable will lose approximately 10% per foot of cable a 300 Watt light source, which results in much lower illumination than desired.

With the introduction of newer minimally invasive surgical techniques, the demand has risen for the delivery of high intensity light through minimal surgical incisions into deep surgical fields. To address this concern, light delivery devices have been developed for delivery of light from a remote, high intensity light sources to the surgical field. The devices consist of bundles of optical fibers that directly adhere to surgical retractors to illuminate the field and are connected via fiber optic cable to a high intensity light source. While these devices provide a technique for directly illuminating the surgical field, they are cumbersome and directly tether the retractors. The fiber bundles are inconvenient. They get in the way and destabilize the retractor positioning. As with head-mounted lighting, they provide highly inefficient illumination.

### SUMMARY

The devices and methods described below provide for improved illumination in the surgical field without the need for fiber bundles and remote power sources. Retractors are provided with light engines mountable on the retractors and means for securing the light engines to the retractors. In various embodiments, the light engines comprise LED's, power sources and control circuitry mounted on the retractor with the LED mounted on the handle portion, wherein light is carried to the tip of the retractor through a fiber optic cable. In other embodiments, the light engines comprise LED's, power sources and control circuitry mounted on the retractor with the LED mounted on the tip of the retractor. The lighting system components may be provided on a substrate that is releasably attachable to the retractors, so that they may be used with various retractors and discarded after use, leaving the retractors to be re-used.

### BRIEF DESCRIPTION OF THE DRAWINGS

FIGS. 1 and 2 illustrate surgical retractors fitted with a light engines.

FIG. 3 illustrates surgical forceps fitted with a light engine and LED.

FIGS. 4 and 5 illustrate a typical surgical field in an open knee, with illuminated retractors in place.

FIG. 6 illustrates a flat blade surgical retractor fitted with a light engines.

FIG. 7 illustrates a light engine adapted for use with the flat blade surgical retractor of FIG. 2.

### DETAILED DESCRIPTION OF THE INVENTIONS

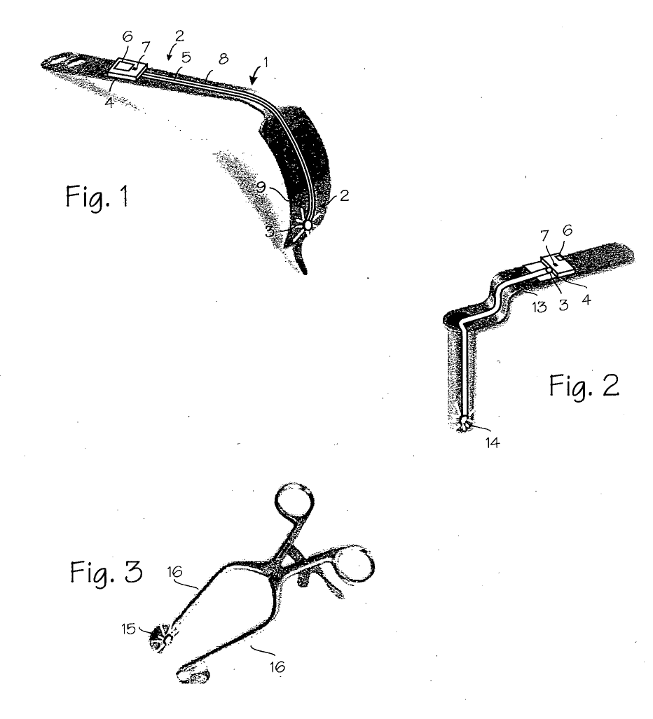

FIG. 1 illustrates a surgical retractor 1 fitted with a light engine 2. The light engine comprises an LED (light emitting diode) 3, battery 4, a conductor 5 electrically connecting the battery and the LED, and an LED control circuit 6 and switch 7 all mounted on a strip of tape 8. The LED is preferably a white-light LED, which provides a bright, white light. The battery may be provided in any form, but is preferably a lithium ion polymer battery provided in small, conformal case. The control circuit, including a switch, is provided in the case or any convenient location. The light engine assembly is mounted on a tape, which may be an adhesive tape or magnetic tape (any other means for releasably securing the battery, conductor and LED to the retractor may be used). The wide blade 9 of this retractor serves as a heat sink for dissipating the heat generated by the LED, and additional heat sinks structures may be added. The assembly may be manufactured and provided separately from the retractor, and packaged separately to enable disposable use of the light engine with non-disposable forceps. The assembly may also be integrated into the retractor, and provided in sterile packaging in kits of retractors assembled to support particular surgeries.

FIG. 2 illustrates a retractor 11 with the light engine 12 comprising an LED 3, battery 4, LED control circuit 6, and switch 7. In this figure, the LED is mounted on the handle portion of the retractor and the light engine further comprises the optical fiber 13 communicating from the LED to the blade portion of the retractor with its output optically communicating with the lens 14. The optical fiber is secured in a channel provided in the retractor. Alternatively, the entire light engine assembly may be secured to the retractor with an adhesive or magnetic tape as described in reference to FIG. 1.

The light engines of FIG. 1 or 2 may also be worn by a surgeon during surgery, by securing the battery and control circuit on the wrist or forearm and securing the LED or lens to the back side of a fingertip.

A variety of instruments may fitted with the light engines of FIGS. 1 and 2, such as the Forceps of FIG. 3, with a lens or LED 15 disposed on one or both of the grasping arms 16 Other instruments may be likewise modified, including dissecting and exploring instruments such as surgical probes, scissors, cannulas, etc. For each embodiment described above, the LED may be powered wirelessly, with the power supply located several feet from the LED.

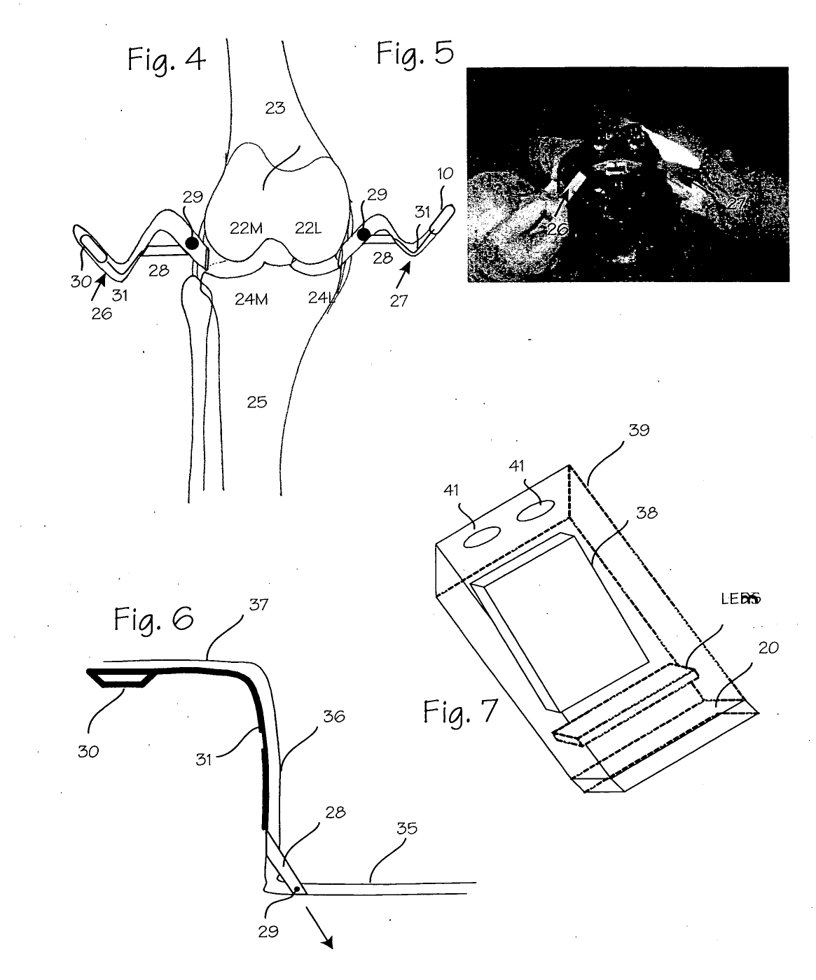

FIGS. 4 and 5 illustrate a surgical field in a knee operation. The patient's knee 21 has been opened an incision, exposing the medial and lateral condyle 22 of femur 23 (the distal femur) and medial and lateral condyle 24 of the fibula 25. The incision is held open with two Z-retractors 26 and 27. A light engine 28 including an LED (light emitting diode) 29, battery 30, a conductor 31 electrically connecting the battery and the LED is disposed on the Z-retractors. The Z-retractors are used as shown, to pry and hold portions of body tissue away from other portions of the joint to maintain an open surgical field. The distal portion is provided with an aperture 33, which accommodates the LED such that light emitted from the LED is directed into the surgical field when the retractor is positioned in the surgical field so as to pry body tissue away from other body tissue such as bones, ligaments and tendons. The LED is preferably a white-light LED, which provides a bright, white light. The battery may be provided in any form, but is preferably a lithium ion polymer battery provided in small, conformal case. The control circuit, including a switch, is provided in the case or any convenient location along the retractor. The light engine assembly may be mounted on a tape, which may be an adhesive tape or magnetic tape (any other means for releasably securing the battery, conductor and LED to the retractor may be used), or it may be manufactured with the retractor so that it is permanently affixed to the retractor. The wide blade form of the distal portion 34 of this retractor serves as a heat sink for dissipating the heat generated by the LED, and additional heat sinks structures may be added.

FIG. 6 shows a side view of the Z-retractor, while FIG. 7 illustrates the light engine. The Z-retractor distal portion 35 is joined at a substantial angel to the intermediate portion 36 of the retractor, and the handle portion 37 is also connected to the intermediate portion at a substantial angle (typically a right angle), disposed opposite the distal portion. The battery is disposed under the handle portion, LED and LED control circuit 38 mounted on the Z-retractor in the crook or recess formed by the lower angle, so that the light engine is opposite the surgical field when in use. The LED and LED control circuit are disposed with a suitable housing 39 adapted to be secured in the crook of the retractor, with the LED or its associated optical window 40 disposed with an aperture in the retractor distal portion such that light from the LED is directed at an angle of about 45° toward the surgical field from distal tip of the distal portion of the retractor. This construction provides for direction of the emitted light toward the surgical field when the Z-retractor is positioned in the typical retracting position in a knee or hip surgery. Electrical connectors 41 provide for connection to the battery, and may be releasably fixed to the conductor 11 to facilitate removal and replacement of the battery and conductor or facilitate complete removal of the lighting system for replacement.

The arrangement illustrated in relation to the Z-retractor may be applied to other flat-bladed retractors, provided that the flat blade retractors are specifically adapted with an aperture and suitable space opposite the surgical field to accommodate the light engine. Other features may be incorporated in the illuminated retractor. The LED light source is preferably recessed or low profile, so that it does not protrude excessively into the surgical field. The optics, if protruding, should be sufficiently durable to withstand impact and abrasion from surgical tools used in the surgical field. The optics or window should be coated with a high temperature non-stick or anti-coagulant coating, especially suited for non-stick and anti-coagulant effect.

Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. An illuminated surgical retractor comprising:
a retractor having a handle portion and a blade portion;
a light emitting diode disposed on the blade portion;
a battery disposed on the handle portion;
a conductor for delivering power from the battery to the LED;
means for releasably securing the battery, conductor and LED to the retractor.

2. An illuminated surgical retractor comprising:
a retractor having a handle portion and a blade portion;
a light emitting diode disposed on the handle portion;
a battery disposed on the handle portion;
a lens disposed on the blade portion;
a optical fiber for delivering light from the LED to the lens;
means for releasably securing the battery, conductor and LED to the retractor.

3. A light engine for use with a surgical retractor comprising:
a light emitting diode adapted for mounting on a blade portion of the retractor;
a battery adapted for mounting on a handle portion of the retractor;
a conductor for delivering power from the battery to the LED;
each of said light emitting diode, battery, and conductor being disposed upon a releasably securable tape for releasably securing the light engine to the retractor.

4. A light engine for use with a surgical retractor comprising:
a light emitting diode adapted for mounting on a handle portion of the retractor;
a battery adapted for mounting on a handle portion of the retractor;
a lens adapted for mounting on a blade portion of the retractor;
a optical fiber for delivering light from the light emitting diode to the lens;
each of said light emitting diode, battery, lens and optical fiber being disposed upon a releasably securable tape for releasably securing the light engine to the retractor.
